(19) United States
(12) Patent Application Publication
(10) Pub. No.: US 2011/0172468 A1
(43) Pub. Date: Jul. 14, 2011

(54) METHOD FOR PRODUCING ALCOHOL, METHOD FOR PRODUCING HYDROGEN OR SYNTHESIS GAS USING THE METHOD FOR PRODUCING ALCOHOL, AND ALCOHOL

(75) Inventors: Hisashi Yamamoto, Yokohama-shi (JP); Naoyoshi Iwasaki, Hitachinaka-shi (JP); Shigeki Nagamatsu, Mito-shi (JP); Yukitoshi Fukuda, Tokyo (JP); Hiromichi Hidaka, Hofu-shi (JP); Atsushi Kouno, Hofu-shi (JP); Yoshihiro Katoh, Hofu-shi (JP)

(73) Assignees: JGC CORPORATION, Chiyoda-ku, Tokyo (JP); KYOWA HAKKO BIO CO., LTD., Chiyoda-ku, Tokyo (JP); KYOWA HAKKO CHEMICAL CO., LTD., Chiyoda-ku, Tokyo (JP)

(21) Appl. No.: 13/062,600

(22) PCT Filed: Sep. 17, 2009
(86) PCT No.: PCT/JP2009/004695

§ 371 Date: Mar. 7, 2011

(30) Foreign Application Priority Data
Sep. 19, 2008 (JP) P2008-241598

(51) Int. Cl. C07C 29/76; C07C 31/02; C07C 29/88

(52) U.S. Cl. 568/917; 568/840; 568/913

(57) ABSTRACT

One object of the present invention is to provide a method for producing an alcohol which can produce a target alcohol containing a remarkably low content of the sulfur compound(s), and the present invention provides a method for producing an alcohol comprising a separation process which reduces the content of sulfur compound(s) in a crude alcohol containing at least the sulfur compound(s) through desulfurization treatment in which the crude alcohol is contacted with a separation membrane based on a pervaporation method, thereby a content of the sulfur composition in the crude alcohol is decreased.

### TECHNICAL FIELD

The present invention relates to a method for producing an alcohol which removes sulfur compound(s) from a crude alcohol containing at least the sulfur compound(s), a method for producing hydrogen or a synthesis gas which uses the method for producing an alcohol, and an alcohol obtained by the method for producing an alcohol.

More specifically, the present invention relates to a method for producing an alcohol which can be used as a raw material for a chemical process including a catalytic reaction, or fuel by selectively removing sulfur compound(s) from a crude alcohol containing at least the sulfur compound(s), a method for producing hydrogen or a synthesis gas which uses the method for producing an alcohol, and an alcohol obtained by this method for producing an alcohol.

The present application claims priority on Japanese Patent Application No. 2008-241598 filed in Japan on Sep. 19, 2008, the content of which is incorporated herein by reference.

### BACKGROUND ART

Alcohols are an important basic materials in the chemical industry, and they can be converted into useful chemical products through various reactions. Alcohols are also used as fuel for internal-combustion engines used in automobiles etc., and other fuels.

Alcohols are mainly produced by chemical reactions from petroleum raw materials, or fermentation from biomass raw materials.

Alcohols, which are obtained by chemical reactions from petroleum raw materials, sometimes contain sulfur compound(s) derived from sulfur compound(s) contained in crude petroleum. In addition, during fermentation, sulfur compound(s) may be generated, and therefore, the sulfur compound(s) may be contained in alcohols which are obtained from biomass raw materials.

When an alcohol contains sulfur compound(s), the sulfur compound(s) are usually separated by desulfurization treatment. Thus, an alcohol can be used, after refinement to a level which does not cause problems, as a raw material of chemical products or various fuels.

Thus, the reasons for desulfurizing and refining alcohols are:
(1) A catalyst used in producing chemical products is poisoned by sulfur compound(s).
(2) When an alcohol containing sulfur compound(s) are burned, a sulfurous acid gas is generated. The sulfurous acid gas causes acid rain unless a special elimination equipment is provided in a combustion equipment for an alcohol. Thus, when an alcohol containing sulfur compound(s) are burned, there exist problems such as that the sulfurous acid gas which has adverse effects on the environment is released into the air.

In addition, when an alcohol is used as fuel in automobiles, there is a problem in which the sulfur compound(s) contained in the alcohol poisons a cleaning catalyst for an exhaust gas.

In a stage of refinement of an alcohol, or a stage of refinement of a crude alcohol obtained by a chemical reaction or fermentation, a composition in which impurities to be separated off are concentrated, is separated together with an alcohol having a desired quality. In many cases, the separated composition contains mainly a target alcohol but also contains sulfur compound(s) and other organic compounds.

Of course, an alcohol containing the sulfur compound(s) and other organic compounds cannot be used as a target alcohol. In addition, such alcohol often contains highly concentrated sulfur compound(s). Therefore, usage applications of the alcohol are restricted. However, the alcohol often contains substantial contents of the target alcohol. Therefore, from the viewpoint of beneficial use of resources, it is important to find a method for removing the adverse effects of the sulfur compound(s) and effectively using it.

From the viewpoint of separating the sulfur compound(s), distillation is one of the effective methods. However, distillation consumes a lot of energy, and has a problem from the viewpoint of saving energy or emission reduction of carbon dioxide.

In addition, in order to obtain an alcohol having target quality with higher yield by distillation, the solutions such as
(1) A yield of the distillation is increased by decreasing a content of distillates having a low boiling point or a high boiling point which are removed;
(2) A distillation column having a higher number of stages is used; and
(3) A content of reflux in the distillation columns is increased are required.

There is an opposite relationship between reduction of the content of distillates to be removed and reduction of the content of the sulfur compound(s) contained in the target distillate by increasing separation efficiency of impurities, such as the sulfur compound(s). Therefore, when the distillation efficiency is increased by using the solution (1), the possibility of contamination of the sulfur compound(s) in the target distillates may be increased. Therefore, this solution (1) naturally has restrictions.

In the solutions (2) and (3), there is a problem of the increase in the construction costs of the distillation columns, and an amount of energy for distillation.

By the way, desulfurization treatment means removal of the sulfur compound(s) contained in the target material by a certain method. In desulfurization processes, a hydrodesulfurization process which desulfurizes petroleum distillates, such as naphtha, gasoline, kerosene, and gas oil, is especially common.

This hydrodesulfurization process is a method of changing the sulfur compound(s) contained in the target material into a compound, such as hydrogen sulfide, by a hydrogenation reaction, and removing that compound by absorption into an adsorbent.

However, when there is an alcohol in the hydrodesulfurization process, a functional group containing an oxygen atom in the alcohol molecule works preferentially on an active site on a hydrodesulfurization treatment catalyst or an adsorbent. Due to this fact, the catalyst or the adsorbent cannot exert ability thereof. In addition, the alcohol itself may be subjected to reaction, depending on the kind of the catalyst or the adsorbent used. Therefore, it is not effective to use a hydrodesulfurization process to remove the sulfur compound(s) from a treated solution containing an alcohol. This problem is caused by the fact that an alcohol contains a functional group having an oxygen atom, dissimilar to the petroleum distillates.

γ-Alumina is widely used as a catalyst support or a molding material of the adsorbent, which are used in a hydrodesulfurization process for petroleum-based materials, because it has a wide specific surface and high stability. However, γ-alumina has high reactivity to an alcohol. Therefore, reactions, such as decomposition of the alcohol, dehydration, dehydrogenation, or polymerization, are promoted. Due to this fact, an alcohol is converted into light hydrocarbon such as methane, ethane, ethylene, or propane, or light hydrocarbon containing an oxygen atom. Therefore, the yield of the target alcohol containing a lower content of sulfur decreases. Thus, γ-Alumina is not preferable when the hydrodesulfurization process is applied to the petroleum-based raw material containing an alcohol.

A method for removing the sulfur compound(s) from an alcohol by an adsorption method is also disclosed (For example, Patent Document No. 1). However, this method uses expensive material such as silver ions. Therefore, this method is not preferably used in industrial application.

### Prior Art Document

### Patent Document

[Patent Document No. 1] PCT International Publication No. WO 2005/063354

### DISCLOSURE OF THE INVENTION

### Problems to be Solved

In consideration of the above-described problems, it is an object of the present invention to provide a method for producing an alcohol comprising a process wherein an alcohol containing a remarkably low content of sulfur compound(s) is obtained by a simple desulfurization treatment from a crude alcohol containing at least sulfur compound(s); a method for producing hydrogen or a synthesis gas which uses the method for producing an alcohol, and an alcohol obtained by the method for producing an alcohol.

### Means for Solving the Problem

The present invention provides a method for producing an alcohol (characterized by) comprising a separation process which reduces the content of sulfur compound(s) in a crude alcohol containing at least the sulfur compound(s) through desulfurization treatment in which the crude alcohol is contacted with a separation membrane based on a pervaporation method.

It is preferable that the separation membrane be one selected from the group consisting of a silicone membrane, a polyimide membrane, a polyamide membrane, a polyester membrane, and a polyvinyl an alcohol membrane.

It is more preferable that the separation membrane be a silicone membrane.

It is preferable that the crude alcohol contain at least one of methanol, 1-propanol, and 2-propanol, and the total content thereof be 1 ppm by weight or more.

It is preferable that the crude alcohol contain 20 ppm by weight or more of methanol, or 200 ppm by weight or more of 1-propanol and/or 2-propanol in total.

It is preferable that the method reduce the total content of sulfur in the crude alcohol to less than 10 ppm by weight.

It is preferable that the method reduce the total content of sulfur in the crude alcohol to less than 1 ppm by weight.

It is preferable that the method reduce the total content of sulfur in the crude alcohol to less than 0.5 ppm by weight.

It is preferable that the crude alcohol contain 10 ppm by weight or more of the sulfur compound(s).

It is preferable that the crude alcohol be diluted with water and applied to the desulfurization treatment.

It is preferable that the crude alcohol be ethanol.

It is preferable that the method include a pretreatment process in which the crude alcohol is subjected to a desulfurization treatment, which is at least one selected from the group consisting of a desulfurization treatment by reaction treatment, a desulfurization treatment by physical adsorption, and a desulfurization treatment using a chemical absorbent, before the separation process.

The present invention also provides a method for producing hydrogen or a synthesis gas, wherein the hydrogen or the synthesis gas is produced by subjecting the alcohol obtained by the method for producing an alcohol according to a present invention to a catalytic reforming reaction

The present invention also provides an alcohol obtained by the method for producing an alcohol according to the present invention.

### Effects of the Present Invention

The method for producing an alcohol of the present invention includes a separation process which reduces the content of sulfur compound(s) in a crude alcohol containing at least the sulfur compound(s) through desulfurization treatment in which the crude alcohol is contacted with a separation membrane based on a pervaporation method. Thereby, an alcohol containing a remarkably low content of sulfur compound(s) from a crude alcohol containing sulfur compound(s), etc. can be obtained by a simple desulfurization treatment.

According to the method of the present invention for producing hydrogen or a synthesis gas, hydrogen or a synthesis gas is produced by subjecting the alcohol obtained by the method for producing an alcohol according to the present invention to a catalytic reforming reaction. Therefore, hydrogen or a synthesis gas can be produced efficiently.

Since the alcohol according to the present invention is obtained by the method for producing an alcohol of the present invention, the total content of the sulfur is less than 10 ppm by weight. Therefore, the alcohol can be used as a raw material for chemical processes containing a catalytic reaction, fuel for automobiles, or other fuels.

### BRIEF DESCRIPTION OF DRAWINGS

FIG. 1 is a schematic view showing one example of a desulfurization equipment used in the method for producing an alcohol according to the present invention.

FIG. 2 is a schematic view showing another example of a desulfurization equipment used in the method for producing an alcohol according to the present invention.

FIG. 3 is a graph showing time course of temperature distribution in an inside reactor chamber when ethanol containing almost no sulfur compound(s) is used in a low temperature steam reforming reaction for ethanol.

FIG. 4 is a graph showing time course of temperature distribution in an inside reactor chamber when ET-1, which is non-desulfurized ethanol shown in Table 2, is used in a low temperature steam reforming reaction for ethanol.

FIG. 5 is a graph showing distribution of an amount of sulfur and carbon attached to a reforming catalyst used in a low temperature steam reforming reaction for ethanol.

### MODE FOR CARRYING OUT THE INVENTION

Best mode for carrying out the method for producing an alcohol, the method for producing hydrogen or a synthesis gas which uses the method for producing the alcohol, and an alcohol obtained by the method for producing an alcohol according to the present invention will be explained.

Moreover, the following embodiments explain the present invention in detail so that the gist of the present invention can be better understood. The present invention is not limited to the following embodiments unless otherwise stated.

First, the alcohol used in the present invention will be explained.

The crude alcohol used in the present invention means mainly a lower alcohol having 2 to 8 carbon atoms, preferably a lower alcohol having 2 to 8 carbon atoms excepting propanols, such as 1-propanol and 2-propanol. Among these, alcohols containing mainly ethanol, butanol, or hexanol is preferable.

The method for producing an alcohol according to the present invention is most preferably used to produce ethanol.

The crude alcohol used in the present invention is not limited depending on the production method thereof. For example, the crude alcohol may be derived from petroleum resources, or biomass resources. In addition, the crude alcohol may contain impurities generated in production processes of the alcohol.

The petroleum-resource raw material, which is used as a raw material, contains sulfur compound(s). Therefore, in the case of the crude alcohol derived from petroleum resources, the obtained alcohol may contain the sulfur compound(s).

Among alcohols, ethanol and butanol are fermentation products which are produced most efficiently in fermentation methods. Therefore, while an environmental problem attracts attention, ethanol and butanol attract attention as a carbon-neutral fuel or a chemical raw material.

The fermentation method is a method in which a raw material is obtained from sugarcane, corn, tapioca, cassava, rice, wheat, waste wood, used paper, etc., and a target product is produced through fermentation processes of these raw materials.

In general, during a fermentation process, are generated sulfur compound(s) which are derived from sulfur contained in amino acids which microorganisms metabolize, or sulfuric acid used in the fermentation process. And there is a possibility that the sulfur or the sulfur compound(s) may be contained in an alcohol. Therefore, the alcohol derived from biomass resources may contain sulfur compound(s).

When such an alcohol containing the sulfur compound(s) is used, the alcohol may act as a catalyst poison to the catalysts used in catalystic reaction processes, or may generate exhaust gas containing toxic substances, such as a sulfurous acid gas. Therefore, since the method for producing an alcohol of the present invention has a process in which the content of sulfur compound(s) in an alcohol is decreased by desulfurization treatment. Such method for producing an alcohol according to the present invention is a valuable method.

In production processes of an alcohol, in order to increase the recovery rate of the alcohol, contamination of a small content of impurities may be permitted as long as practical problems are not caused.

The alcohol produced by these production processes may contain sulfur compound(s) even when it is refined. This alcohol is the representative example of “an alcohol containing sulfur compound(s)” in the present invention.

When the purpose of using an alcohol changes, the sulfur compound(s) contained in the alcohol may cause a problem. The scope of the present invention includes a method for producing an alcohol containing a less content of the total sulfur compound(s) by desulfurizing the alcohol obtained by these production processes; an alcohol having the decreased total content of the sulfur compound(s); and a method for producing hydrogen or a synthesis gas by using the alcohol having the decreased total content of the sulfur compound(s).

In general, an alcohol is refined via a distillation process. In this case, a compound having a relative volatility which is similar to that of the alcohol causes a problem. When an alcohol is produced by chemical reactions, water produced in the reactions often coexists with the alcohol in a refining process. In addition, when an alcohol is produced by fermentation, water used in the fermentation process often coexists with the alcohol in a refining process. Therefore, as long as water coexists with the alcohol in a distillation process, the relative volatility at a concentration range of the alcohol containing water has to be considered.

By the way, methanol and propanols are the homologous compounds of ethanol, butanol, and the like. Due to this fact, when ethanol, butanol, or the like is produced, methanol or propanols are often produced at the same time.

In general, when ethanol or butanol is refined by distillation, methanol or propanols contaminated are removed as a lower boiling point distillate or higher boiling point distillate than that of ethanol or butanol. However, methanol or propanols have a concentration range in which the relative volatility to ethanol or butanol is small in an aqueous system. Therefore, it is difficult to remove methanol or propanols from ethanol or butanol. As a result, the distillate separated from the crude alcohol may contain a substantial content of ethanol or butanol together with much propanols or methanol.

Here, “propanols” means 1-propanol and 2-propanol.

As explained above, the sulfur compound(s) may be derived from a petroleum-resources raw material or generated in fermentation processes. Among these sulfur compound(s), the sulfur compound(s) which have a small relative volatility to ethanol or butanol preferably contained in the crude alcohol especially causes the problems in the present invention.

Similar to methanol or propanols explained above, the sulfur compound(s) are removed as a lower boiling point distillate or higher boiling point distillate than that of ethanol or butanol preferably contained in the crude alcohol.

In other words, when ethanol or butanol is the target alcohol to be produced in the present invention, there are distillates which are separated as a lower boiling point distillate or higher boiling point distillate from the distillate which satisfy the quality of the target alcohol, together with the distillate of the target alcohol. These distillates contain methanol or propanols, and the sulfur compound(s) contained in the alcohol prior to refinement. As explained above, it is difficult to separate these methanol or propanols, and the sulfur compound(s) from the target alcohol. Therefore, these distillates contain methanol, or propanols, and the sulfur compound(s) in addition to a substantial content of the target alcohol. That is, it is possible to say that according to a conventional method for producing an alcohol, an alcohol distillate having a low purity is obtained.

This alcohol distillate having a low purity is a representative example of “the crude alcohol containing the sulfur compound(s) or the crude alcohol containing the sulfur compound(s) and 1 ppm by weight or more of methanol or propanols” in the present invention.

In the present invention, “total content of sulfur” means the total content of compounds containing sulfur contained in the crude alcohol, and the total content of compounds containing sulfur is expressed in terms of the weight percent of sulfur. When an alcohol is diluted with water, the total content of compounds containing sulfur contained in the alcohol before dilution is expressed in terms of the weight percent of sulfur.

The crude alcohol, which is the raw material in the production method according to the present invention, contains a large content of sulfur compound(s) which generate poison for catalyst or a sulfurous acid gas. Therefore, it is difficult to use the crude alcohol in industrial applications unless the sulfur compound(s) are removed.

Examples of the sulfur compound(s) contained in the crude alcohol include sulfides, such as dimethyl sulfide, diethyl sulfide, ethyl methyl sulfide, and dibutyl sulfide; disulfides such as dimethyl disulfide, diethyl disulfide, ethyl methyl disulfide and dibutyl disulfide; thiocarboxylic acids such as methyl thioacetate, and S-methylthioacetic acid; aromatic sulfur compound(s) such as thiophene, methylthiophene, benzthiophene; sulfites such as dimethyl sulfite, diethyl sulfite, and dibutyl sulfite; sulfates such as dimethyl sulfate, diethyl sulfate, and dibutyl sulfate, and the like.

Below, the method for producing an alcohol according to the present invention is explained in detail.

### First Embodiment of the Method for Producing an Alcohol

The first embodiment of the method for producing an alcohol according to the present invention is a method for producing an alcohol (characterized by) comprising a separation process which reduces the content of sulfur compound(s) in a crude alcohol containing at least the sulfur compound(s) through desulfurization treatment in which the crude alcohol is contacted with a separation membrane based on a pervaporation method, and a content of the sulfur compound(s) in the crude alcohol is decreased.

It is thought that the desulfurization treatment by contacting with the separation membrane used in the present invention is proceeded by mechanisms based on the pervaporation method.

In the present invention, the pervaporation method is a membrane separation method in which when the crude alcohol to be treated is passing through the separation membrane from the supply side to the permeation side (recovery side), a compound which is contained in the crude alcohol and has a high affinity to the separation membrane is removed by evaporation, and thereby the correspondent compound is removed from the crude alcohol.

Specifically, when the separation membrane has a high affinity to the sulfur compound(s), the sulfur compound(s) in the crude alcohol containing the sulfur compound(s) selectively permeate through the separation membrane from a supply side, and evaporate to move into a permeations side, that is a recovery side. As a result, desulfurized an alcohol, which does not pass through the separation membrane, remains in the supply side.

In general, the pervaporation method denotes a method in which a liquid phase is evaporated via the separation membrane. However, the present invention includes not only a case in which an alcohol is supplied in a liquid phase, but also a case in which an alcohol is supplied in a vapor phase or a vapor-liquid mixed phase, to contact with the separation membrane.

It is considered that when an alcohol containing the sulfur compound(s) in a vapor-liquid mixed phase is desulfurized by supplying to the separation membrane by mechanisms based on the pervaporation method, the sulfur compound(s) in a vapor phase permeate selectively through the separation membrane to the opposite side of the separation membrane in addition to the sulfur compound(s) in a liquid phase permeate selectively through the separation membrane to the opposite side of the separation membrane.

In the desulfurization treatment in which the crude alcohol containing the sulfur compound(s) is in contact with the separation membrane based on a pervaporation method, the flow rate of the alcohol supplied to the supply side of the separation membrane is preferably in a range of 0.01 cm/second to 300 cm/second, more preferably in a range of 0.05 cm/second to 150 cm/second, and most preferably in a range of 0.1 cm/second to 50 cm/second as an average linear velocity.

When the average linear velocity is 0.01 cm/second or more, a long period of time is not required to desulfurize. In contrast, when the average linear velocity is 300 cm/second or less, the desulfurization treatment is carried out using a common equipment without an expensive equipment, as well as a preferable efficiency of the desulfurization treatment being obtained.

In the desulfurization treatment in which the crude alcohol containing the sulfur compound(s) is in contact with the separation membrane based on the pervaporation method, the pressure to supply the crude alcohol in the supply side of the separation membrane is not particularly limited, and preferably adjusted depending on flow rate of the crude alcohol and characteristics of the desulfurization equipment.

However, the pressure of the crude alcohol containing the sulfur compound(s) which is supplied to the supply side of the separation membrane during the desulfurization treatment is preferably in a range of 10 kPa to 10 MPa, more preferably in a range of 10 kPa to 1 MPa, and most preferably in a range of 50 kPa to 0.5 MPa.

When the pressure of the crude alcohol containing the sulfur compound(s) is 10 kPa or more, a suitable evaporation rate by the pervaporation method can be obtained, and a long period of time is not necessary to desulfurize. In contrast, when the pressure of the crude alcohol containing the sulfur compound(s) is 10 MPa or less, a content of an alcohol evaporated by passing through the separation membrane is adequate, and as a result a suitable desulfurization treatment efficiency can be maintained. In addition, an equipment having a high pressure resistance is not necessary, and a common equipment can be used to desulfurize.

In the desulfurization treatment in which the crude alcohol containing the sulfur compound(s) is in contact with the separation membrane based on the pervaporation method, the pressure at the permeation side of the separation membrane (that is, the pressure at the recovery side or the pressure in the opposite side to the supply side relative to the separation membrane) is not particularly limited as long as it is adjusted to the pressure in the supply side or less. However, the difference in pressure between the recovery side and the supply side of the separation membrane is preferably in a range of 0 kPa to 10 MPa, more preferably in a range of 0.05 kPa to 1 MPa, and most preferably in a range of 0.1 kPa to 0.5 MPa. In other words, it is preferable that the pressure in the recovery side of the separation membrane be adjusted so as to be lower than the pressure of the supply side by a range of 0 kPa to 10 MPa, more preferably a range of 0.05 kPa to 1 MPa, and most preferably a range of 0.1 kPa to 0.5 MPa.

When the difference in pressure between the recovery side and the supply side is 0 kPa or more, the sulfur compound(s) easily passes through the separation membrane from the supply side to the recovery side by the pervaporation method. In contrast, when the difference in pressure is 10 MPa or less, the separation membrane is not required to have a high pressure resistance. Therefore, the structure of the separation membrane and a supporter is simple, and an expensive desulfurization equipment is not necessary. In addition, a thick separation membrane is not necessary. Due to this, high desulfurization treatment efficiency can be obtained.

In the desulfurization treatment in which the crude alcohol containing the sulfur compound(s) is in contact with the separation membrane based on the pervaporation method, the temperature of the crude alcohol containing the sulfur compound(s) supplied in the supply side of the separation membrane is preferably in a range of 0° C. to 100° C., more preferably in a range of 10° C. to 70° C., and most preferably in a range of 20° C. to 50° C.

When the temperature of the crude alcohol containing the sulfur compound(s) is 0° C. or more, a suitable evaporation rate can be maintained, and therefore the time for desulfurization treatment can be shortened. In contrast, when the temperature of the crude alcohol containing the sulfur compound(s) is 100° C. or less, a content of the target alcohol obtained by passing through the separation membrane to evaporate can be adjusted to be adequate amount. Due to this, not only a high recovery ratio of the target alcohol but also high desulfurization treatment efficiency can be maintained.

Since the sulfur compound which passed through the separation membrane is vapor (in a gas state), the sulfur compound(s) in a gas state can be collected in a trap vessel in the permeation side (recovery side) of the separation membrane, while at the same time, the sulfur compound(s) collected are cooled by a cooling equipment or liquid nitrogen, and thereby the sulfur compound(s) are recovered as liquid. At this time, a part of an alcohol passed through the separation membrane may be recovered.

It is preferable that the temperature to cool the sulfur compound(s) in a gas state collected in the trap vessel be adjusted to lower than the boiling point under the pressure in the permeation side (recovery side) of the separation membrane by about 30° C. When the temperature is adjusted so as to satisfy this condition, collection efficiency of the sulfur compound(s) is high, and excess energy to cool the sulfur compound(s) in a gas state is not necessary.

The separation membrane is one selected from a silicone membrane, a polyimide membrane, a polyamide membrane, a polyester membrane, and a polyvinyl an alcohol membrane. Among these, from the viewpoint of ease of availability, and excellent selective permeation properties of the sulfur compound(s), a silicone membrane is preferable.

Here, the silicone membrane is a generic name of separation membranes made of silicone in the present invention. Similarly, the polyimide membrane is a generic name of separation membranes made of polyimide. The polyamide membrane is a generic name of separation membranes made of polyamide. Similarly, the polyester membrane is a generic name of separation membranes made of polyester. The polyvinyl an alcohol membrane is a generic name of separation membranes made of polyvinyl an alcohol.

As long as the crude alcohol containing the sulfur compound(s) can be in contact with separation membrane, any type of the separation membrane can be used without any limitation. For example, one type selected from the group consisting of a hollow fiber type, a tube type, a flat type, a capillary tube type, a spiral type, and a pipe type, can be used.

First, the hollow fiber type separation membrane is explained.

The hollow fiber type separation membrane is a separation membrane obtained by binding a lot of number of long hollow fibers having a straw shape, or a macaroni shape.

In the membrane separation of the crude alcohol containing the sulfur compound(s) using the hollow fiber type separation membrane, the crude alcohol is flown (passed) through the inside of the hollow fiber membranes while adding pressure to the crude alcohol. In other words, the separation treatment of the sulfur compound(s) contained in the crude alcohol, that is, desulfurization treatment, is carried out by making the crude alcohol pass through the hollow fiber membranes from the inside to the outside.

It is preferable that the inner diameter of the hollow fiber membranes constituting the hollow fiber type separation membrane be in a range of 0.01 mm to 100 mm, more preferably in a range of 0.01 mm to 30 mm, and most preferably in a range of 0.1 mm to 5 mm.

When the inner diameter of the hollow fibers is 0.01 mm or more, a suitable content of the crude alcohol containing the sulfur compound(s) can be desulfurized. In addition, it is not necessary to apply high pressure to the supply side of the separation membrane. In contrast, when the inner diameter of the follow fiber membranes is 100 mm or less, the contact efficiency between the crude alcohol and the separation membrane is improved. As a result, high desulfurization treatment efficiency can be maintained.

The outer diameter of the hollow fiber membranes which constitute a hollow fiber type separation membrane is determined based on the preferable inner diameter of the hollow fibers and a thickness of the separation membrane. Therefore, the outer diameter of the hollow fibers is not particularly limited. However, the outer diameter of the hollow fibers is preferably in a range of 0.01 mm to 100 mm, more preferably in a range of 0.01 mm to 50 mm, and most preferably in a range of 0.1 mm to 10 mm. The outer diameter of the hollow fiber membranes is never smaller than the inner diameter of the hollow fiber membranes.

When the outer diameter of the hollow fiber membranes is 0.01 mm or more, a suitable content of the crude alcohol containing the sulfur compound(s) can be desulfurized and it is not necessary to apply high pressure to the supply side of the separation membrane. In contrast, when the diameter of the hollow fibers is 100 mm or less, the contact efficiency between the crude alcohol and the separation membrane is improved. As a result, a high desulfurization treatment efficiency can be maintained.

An effective length of the hollow fiber type separation membrane can be adjusted depending on the flow rate of the crude alcohol containing the sulfur compounds supplied in the supply side of the separation membrane. However, the effective length of the hollow fibers is preferably in a range of 1 cm to 300 cm, more preferably in a range of 5 cm to 200 cm, and most preferably in a range of 10 cm to 150 cm.

When the effective length of the hollow fiber membrane is 1 cm or more, suitable contacting time between the crude alcohol and the separation membrane can be maintained, and thereby, a high desulfurization treatment efficiency can be obtained. In contrast, when the effective length of the hollow fiber membrane is 300 cm or less, the size of the desulfurization equipment is not required to be large. Due to this, it is preferable from the view point of industrial applications.

Here, the effective length of the hollow fiber membrane is a length of hollow fiber membrane which works actually as a separation membrane the crude alcohol containing the sulfur compound(s).

A number of the hollow fiber membranes constituting the hollow fiber type separation membrane is adjusted depending on the flow rate of the crude alcohol containing the sulfur compounds supplied in the supply side of the separation membrane. However, the number of the hollow fiber membranes is preferably in a range of 2 to 30,000, more preferably in a range of 100 to 10,000, and most preferably in a range of 1,000 to 8,000,

When the number of the hollow fiber membranes is 2 or more, a suitable content of the crude alcohol can be desulfurized. In contrast, when the number of the hollow fiber membranes is 30,000 or less, the size of the desulfurization equipment is not required to be large. Due to this, it is preferable from the view point of industrial applications.

An area in the hollow fiber type separation membrane, at which the crude alcohol containing the sulfur compound(s) contacts, that is, a total area of the inner walls of all the hollow fiber membranes constituting the separation membrane (below, abbreviated as “total surface area of the hollow fiber type separation membrane”), is adjusted depending on the structure of the preferable separation membrane, and this is not particularly limited.

However, the total surface area of the hollow fiber type separation membrane is preferably in a range of 0.01 m² to 100 m², more preferably in a range of 0.02 m² to 50 m², and most preferably in a range of 0.03 m² to 10 m².

When the total surface area is 0.01 m² or more, a high desulfurization treatment efficiency can be obtained. In contrast, when the total surface area is 100 m² or less, the desulfurization equipment is easily designed.

Next, the tube type separation membrane is explained.

A tube type separation membrane is a long tube separation membrane.

In the membrane separation of an alcohol using this tube type separation membrane, the sulfur compound(s) included in the crude alcohol are removed by passing the crude alcohol to be desulfurized in the tube, and evaporating from the inside to the outside of the tube.

The effective length of the tube in the tube type separation membrane is determined depending on the residence time and treatment rate in the desulfurization treatment of the crude alcohol containing the sulfur compound(s) in the tube needed for desulfurization treatment, and it is not particularly limited. However, the effective length of the tube is preferably in a range of 1 m to 1,000 m, more preferably in a range of 2 m to 500 m, and most preferably in a range of 4 m to 100 m.

When the effective length of the tube is 1 m or longer, sufficient residence time of the crude alcohol in the tube for desulfurization treatment can be maintained. In contrast, when the effective length of the tube is 1,000 m or less, a suitable desulfurization treatment time can be obtained. In addition high pressure is not necessary to pass the crude alcohol through the tube.

Here, the effective length of the tube in the tube type separation membrane means a length in which the crude alcohol containing the sulfur compound(s) can be membrane-separated actually, not a total length of the tube type separation membrane.

Preferable conditions of the tube type separation membrane, such as an inner diameter, an outer diameter, and a total surface area are the same as those in the hollow fiber type separation membrane.

Next, a flat type separation membrane is explained.

The flat type separation membrane is a separation membrane in which a pair of two separation membranes are arranged with a fixed interval so as to face each other. In this type of separation membrane, a spacer is arranged between the membranes in the supply and the permeation sides, and a flow channel for the crude alcohol containing the sulfur compound(s) is formed between the membranes in the supply and the permeation sides.

In the separation membrane for the crude alcohol using the flat type separation membrane, the desulfurization treatment is carried out by flowing (passing) the crude alcohol containing the sulfur compound(s) parallel to the separation membranes while applying pressure, permeating the sulfur compounds) contained in the crude alcohol to the outsides of the separation membranes to remove.

Next, the capillary type separation membrane is explained.

The capillary type separation membrane is a separation membrane which does not need a supporter, and has basically the same structure as that of the hollow fiber type separation membrane. The difference between the capillary type separation membrane and the hollow fiber type separation membrane is that the size of the capillary type separation membrane is smaller than the size of the hollow fiber type separation membrane.

In the separation membrane for the crude alcohol using the capillary type separation membrane, similarly to the hollow fiber type separation membrane, the desulfurization treatment is carried out by flowing (passing) the crude alcohol containing the sulfur compound(s) inside of the capillary while applying pressure, permeating the sulfur compound(s) contained in the crude alcohol from the inside to the outsides of the capillary.

Then, the spiral type separation membrane is explained.

The spiral type separation membrane is a separation membrane which is obtained by rolling the flat type separation membrane like a sandwich roll.

In the separation membrane for the crude alcohol using the spiral type separation membrane, similarly to the flat type separation membrane, the desulfurization treatment is carried out by flowing (passing) the crude alcohol containing the sulfur compound(s) parallel to the separation membrane while applying pressure, permeating the sulfur compound(s) contained in the crude alcohol from the inside to the outside of the separation membrane to remove.

Lastly, the pipe type separation membrane is explained.

The pipe type separation membrane needs a supporter. For example, the supporter may be a porous stainless steel pipe, a ceramics pipe, or a plastic pipe, and the supporter is arranged inside of the membrane.

In the separation membrane for the crude alcohol using the pipe type separation membrane, similarly to the hollow fiber type separation membrane, the desulfurization treatment is carried out by flowing (passing) the crude alcohol containing the sulfur compound(s) inside of the pipe while applying pressure, permeating the sulfur compound(s) contained in the crude alcohol from the inside to the outside of the pipe.

As explained above, it is preferable that the crude alcohol containing the sulfur compounds be diluted with water, and supplied for the desulfurization treatment.

In general, when the alcohol is diluted with water, dilution heat generates. Therefore, a part of the sulfur compound(s) having a low boiling point evaporates, and the total content of the sulfur compound(s) contained in the crude alcohol decreases. However, it is difficult to desulfurize the alcohol so as to achieve the level of using as a raw material for chemical processes including a catalytic reaction or fuel for automobiles by only dilution.

In the present invention, the desulfurization treatment efficiency may be further improved by supplying the crude alcohol containing the sulfur compound(s) diluted with water rather than the crude alcohol containing the sulfur compound(s) without dilution.

The reason is considered that the affinity between the alcohol and the separation membrane changes due to water. That is, water easily makes interaction to the alcohol by hydrogen bonds. Therefore, when water exists, the affinity between the alcohol and the separation membrane decreases. As a result, it is suggested that the affinity between the sulfur compound(s) and the separation membrane is larger than the affinity between the alcohol and the separation membrane.

In addition, the recovery ratio of the alcohol is further improved by supplying the crude alcohol containing the sulfur compound(s) diluted with water rather than the crude alcohol containing the sulfur compound(s) without dilution.

The reason is considered that an alcohol easily makes aggregates by water using hydrogen bonds. When the aggregates are formed, the alcohol become harder to evaporate. Due to this, the amount of the alcohol evaporated in the recovery side of the separation membrane based on the pervaporation method is decreased.

When diluting the crude alcohol containing the sulfur compound(s) with water, the content of water added in the diluted an alcohol is preferably in a range of 20% by weight to 80% by weight, more preferably in a range of 30% by weight to 60% by weight, and most preferably in a range of 40% by weight to 50% by weight.

When the content of water is 20% by weight or more, a sufficient content of the aggregates between the alcohol and water is formed. Thereby, the alcohol does not readily evaporate, and the recovery ratio of the alcohol can be improved. In contrast, when the content of water is 80% by weight or less, the size of the desulfurization equipment is not required to be large. In addition, it is not necessary to separate water from the alcohol after desulfurization treatment in the next process.

In addition, when the crude alcohol containing the sulfur compounds is diluted with water, water may be added just before the desulfurization treatment. Furthermore, it is also possible to use an alcohol which is originally diluted with water, such as a fermentation solution.

Below, the desulfurization treatment of an alcohol using the desulfurization equipment including the hollow fiber type separation membrane is explained in more detail.

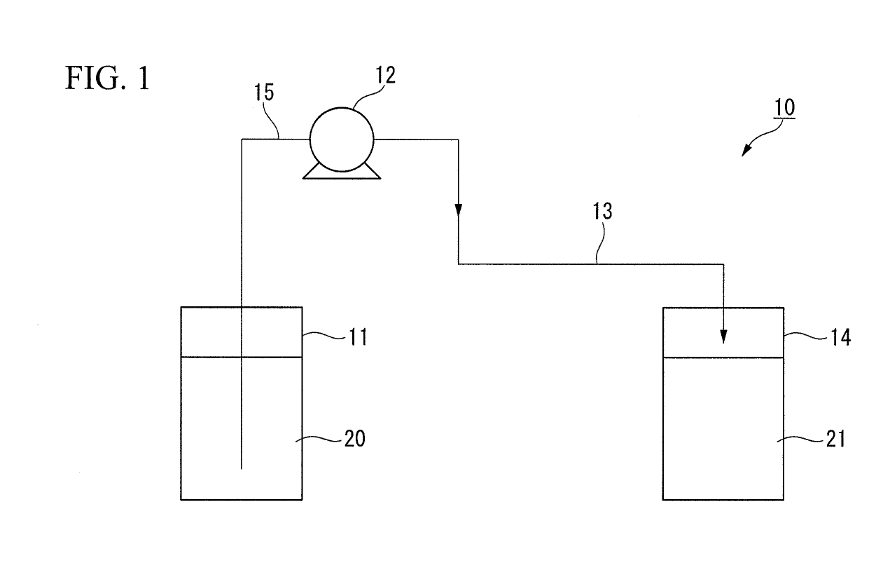

FIG. 1 is a schematic view showing one example of a desulfurization equipment used in the method for producing an alcohol according to the present invention.

The desulfurization equipment 10 shown in FIG. 1 includes a vessel 11 for storing the crude alcohol 20 containing the sulfur compound(s), a pump 12 for sending the crude alcohol 20, a tube type separation membrane 13, a recovery vessel 14 for recovering a desulfurized solution 21 treated in the separation membrane 13, a flow channel 15 for connecting between the vessel 11 and the separation membrane 13.

In this desulfurization equipment 10, when non-desulfurized an alcohol 20 in the vessel 11 is supplied to the tube type separation membrane 13 via the flow channel 15, during passing through the separation membrane 13, the sulfur compound(s) contained in the alcohol 20 is evaporated to the outside of the separation membrane 13 by pervaporation. Thereby, the target alcohol containing a less content of the sulfur compound(s) is sent to the recovery vessel 14.

With a separation membrane other than the tube type separation membrane, the separation membrane is provided instead of the separation membrane 13 in the desulfurization equipment 10.

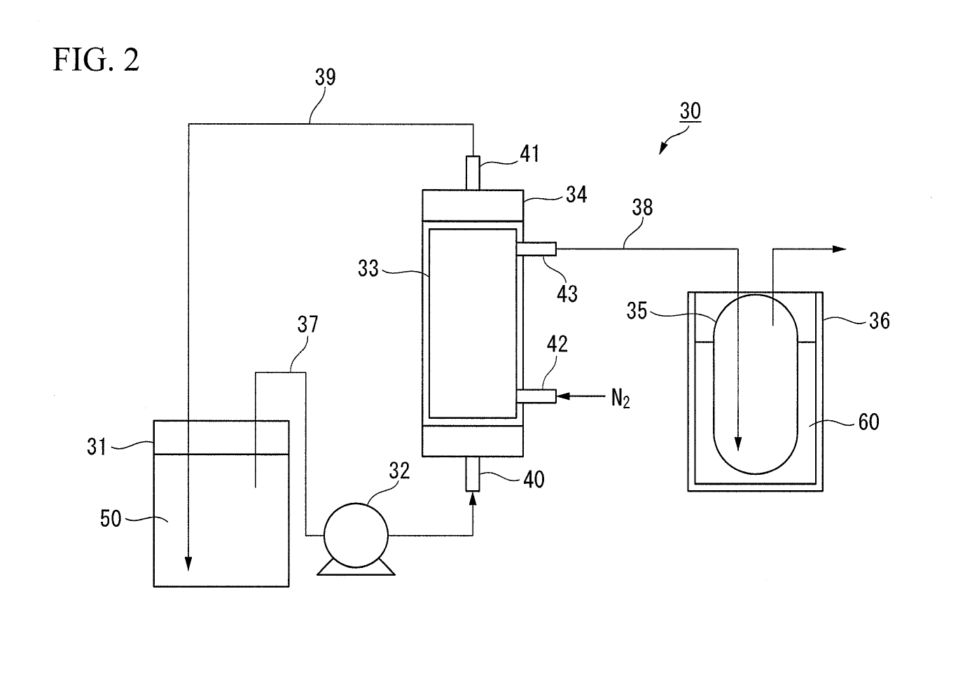

FIG. 2 is a schematic view showing another example of a desulfurization equipment used in the method for producing an alcohol according to the present invention.

The desulfurization equipment 30 shown in FIG. 2 includes a vessel 31 for storing the crude alcohol 50 containing the sulfur compound(s), a pump 32 for sending the crude alcohol 50, a separator 34 including the hollow fiber type separation membrane 33 (below, abbreviated as “hollow fiber type separation membrane”) obtained by binding a lot of number of hollow fiber membranes, a trap vessel 35 for trapping the sulfur compound(s) in a gas state separated in the separator 34, a low-temperature storing thermal insulation vessel 36 for storing liquid nitrogen 60, which is used to cool the sulfur compound(s) in a gas state trapped in the trap vessel 35, and flow channels 37, 38, and 39.

In this desulfurization equipment 30, the crude alcohol 50 containing the sulfur compound(s) in the vessel 31 is sent by the pump 32 to the supply side of the hollow fiber type separation membrane 33 in the separator 34 from an inlet 40 through the flow channel 37. Then, while passing through the hollow fiber type separation membrane 33, the sulfur compound(s) contained in the alcohol 50 evaporates by the pervaporation to the outside of the hollow fiber type separation membrane 33.

After that, the evaporated sulfur compound(s) are discharged toward the outside of the separator 34 from the outlet 43, and sent to the trap vessel 35 through the flow channel 38.

Then, the sulfur compound(s) sent into the trap vessel 35 are cooled by the liquid nitrogen 60 in the low-temperature storing thermal insulation vessel 36, liquefied, and recovered.

Here, nitrogen gas is sent to the outside of the hollow fiber type separation membrane 33 in the separator 34 from the gas inlet 42.

In addition, as explained above, since the sulfur compound(s) are separated by the hollow fiber type separation membrane 33, the content of the sulfur compound(s) in the crude alcohol 50, which does not permeate through the hollow fiber type separation membrane 33, also decreases. In other words, the crude alcohol 50 contains the target alcohol with a high concentration. Therefore, the treated alcohol 50 contains the sulfur compound(s) with a low concentration and the target alcohol with a high concentration, is discharged to the outside of the separator 34 from the outlet 41, and returned to the vessel 31 through the flow channel 39.

As explained above, by using pump 32 the crude alcohol 50 containing the sulfur compound(s) is circulated in a circuit of the vessel 31→the flow channel 37→the separator 34→the flow channel 39→the vessel 31→the flow channel 37→ . . . . At the same time, a part of the sulfur compound(s) permeates through the hollow fiber type separation membrane 33, and the crude alcohol 50 is desulfurized.

In the first embodiment of the method for producing an alcohol of the present invention, the crude alcohol containing the sulfur compound(s) or the crude alcohol containing the sulfur compound and 1 ppm by weight or more of methanol or propanols is desulfurized by contacting to the separation membrane based on the pervaporation method. It is possible to decrease the total sulfur content in the treated an alcohol preferably to less than 10 ppm by weight, more prefrerably to less than 1 ppm by weight, and most preferably to less than 0.5 ppm by weight. Therefore, it is possible to produce an alcohol, which can be used as a raw material for a chemical process containing a catalytic reaction, fuel for automobiles, and other fuels.

### Second Embodiment of the Method for Producing an Alcohol

In the second embodiment of the method for producing an alcohol according to the present invention, prior to the separation in the first embodiment explained above, the crude alcohol containing the sulfur compound(s) is subjected to at least one pre-desulfurization treatment selected from a desulfurization treatment by reaction process, a desulfurization treatment by physical adsorption, and desulfurization treatment by a chemical absorbent.

That is, the difference between the first and the second embodiments of the method for producing an alcohol is that after the pre-desulfurization treatment selected from a desulfurization treatment by reaction process, a desulfurization treatment with a physical absorbent, and desulfurization treatment with a chemical absorbent, is carried out, the desulfurization treatment using the separation membrane based on the same pervaporization method as that in the first embodiment, is carried out.

The desulfurization treatment by reaction process as the pre-desulfurization treatment is a treatment in which the sulfur compound(s) is converted to a compound having-different properties from those of the sulfur compound(s) by a chemical reaction, and the obtained compound is removed by any method. The most ordinary method among the desulfurization treatment method by reaction process is hydrodesulfurization. The hydrodesulfurization is a method in which the sulfur compound(s) is converted to hydrogen sulfide by a hydrogenation reaction (hydrogen addition reaction), and the hydrogen sulfide is removed by being adsorbed in an adsorbent.

Specifically, in the present invention, the hydrogenation reaction (hydrogen addition reaction) is a reaction in which the crude alcohol containing sulfur compound(s) is contacted with a catalyst in the presence of hydrogen.

By the hydrogenation reaction, the sulfur compound(s) is converted to hydrogen sulfide. Therefore, it is possible to remove the hydrogen sulfide by being adsorbed in an adsorbent.

In the method for producing an alcohol according to the present invention, it is preferable that the catalyst used in the hydrogenation reaction (hydrogen addition reaction) be supported on a support.

As the support for a catalyst used in the hydrogenation reaction, is preferable a support such that a yield of ethanol is 60% or more when pure ethanol is contacted with such support at 370° C. at normal pressure.

In addition, it is preferable that the content of α-alumina contained in the support be less than 3% by weight.

Examples of the support include at least one selected from the group consisting of silica (SiO₂), titania (TiO₂), activated carbon (ACTIVATED CARBON, AC), magnesia (MgO), and α-alumina (α-Al₂O₃).

Examples of the catalyst used in the hydrogenation reaction include at least one selected from the group consisting of nickel (Ni), molybdenum (Mo), cobalt (Co), platinum (Pt), palladium (Pd), ruthenium (Ru), and rhodium (Rh). Specifically, a Co—Mo type loaded oxide catalyst, a Ni—Mo type loaded oxide catalyst, a Pd loaded activated carbon, a Pt loaded activated carbon, etc. can be used.

The temperature in the hydrogenation reaction is preferably in a range of 0° C. to 400° C., and more preferably in a range of 100° C. to 300° C.

When the temperature in the hydrogenation reaction is in a range of 0° C. to 400° C., the yield of the target alcohol containing the sulfur compound(s) with a low concentration can be further improved.

In addition, the reaction pressure in the hydrogenation reaction is preferably in a range of normal pressure to 5 MPaG, and more preferably in a range of normal pressure to 3 MPaG.

When the pressure in the hydrogenation reaction is in a range of normal pressure to 5 MPaG, the content of light hydrocarbon gases, such as methane and ethane decreases. Thereby, not only the yield of the alcohol containing the sulfur compound(s) with a low concentration, but also the design pressure of the reaction equipment can be decreased. The cost for the reaction equipment is lowered, and this is economical.

As the support for a catalyst used in the hydrogenation reaction, is preferable a support such that a yield of ethanol is 60% or more when pure ethanol is contacted with such support at 370° C. at normal pressure.

In addition, it is preferable that the content of y-alumina contained in the adsorbent be less than 3% by weight.

Examples of the adsorbent include adsorbents which contain at least one selected from the group consisting of zinc compounds, such as zinc oxide, and iron compounds such as iron oxide, and the total content of these compounds is 30% by weight or more.

In addition, the adsorbent may contain at least one selected from the group consisting of silica, titania, magnesia, and alumina, and contains less than 3% by weight of γ-Al₂O₃.

The following method can also be used other than the method using the hydrogenation reaction, as desulfurization treatment by reaction process.

The crude alcohol containing the sulfur compound(s) is in contact with an ion-exchange resin or a solid catalyst. Specifically, the method is (1) a method in which the crude alcohol containing the sulfur compound(s) continuously flows in a tower filled with an ion-exchange resin or a solid catalyst, and (2) an ion-exchange resin or a solid catalyst, and the crude alcohol containing the sulfur compound(s) are put in a batch type reactor, and stirred to contact each other.

When the crude alcohol containing the sulfur compound(s) is in contact with an ion-exchange resin or a solid catalyst to desulfurize, the sulfur compound(s) contained in the crude alcohol is adsorbed chemically to the ion-exchange resin or the solid catalyst, and thereby the sulfur compound(s) are removed from the crude alcohol. Due to this, the crude alcohol is desulfurized.

However, some ion-exchange resins and solid catalysts also have a property such that sulfur compound(s) are converted into compound(s) whose property is different from that of alcohol. Therefore, depending upon compounds, it is possible that sulfur compound(s) subjected to the above conversion reaction are separated from alcohol and that as a result the alcohol is desulfurized.

In addition, some ion-exchange resins or solid catalyst may adsorb physically the sulfur compound(s). Some sulfur compound(s) may be desulfurized by a physical adsorbent.

In other words, in the desulfurization treatment in which the crude alcohol is contacted with an ion-exchange resin or a solid catalyst, it is important to contact the ion-exchange resin or the solid catalyst with the crude alcohol containing sulfur compound(s) and the desulfurization treatment is not limited to the desulfurization using a chemical absorbent, and may contain the desulfurization by reaction treatment or by a physical adsorbent.

As the ion-exchange resin, at least one of a cation-exchange resin and an anion-exchange resin can be used in the present invention.

As the solid catalyst, activated white clay, heteropoly acid, silica, alumina, or zeolite can be used.

In the methods (1) and (2), the temperature (it may be called “contact temperature” below) when the crude alcohol containing the sulfur compounds is in contact with the ion-exchange resin or the solid catalyst is preferably in a range of 0° C. to 200° C., and more preferably in a range of room temperature (25° C.) to 100° C.

The contact temperature is preferably in a range of 0° C. to 200° C., because a dehydration reaction or a condensation reaction of the alcohol, which is caused by catalyst functions of the ion-exchange resin or the solid catalyst, does not readily occur in the contact temperature range.

In addition, the methods (1) and (2) can be carried out under pressure depending on the contact temperature.

In addition, plural ion-exchange resins or solid catalysts can be used at the same time.

The sulfur compound(s) whose properties are converted to those different from alcohol is generally separated by distillation, adsorption, etc.

In addition, when the boiling point of the sulfur compound(s) having the changed properties is sufficiently low, the sulfur compound(s) having the changed properties can be removed as gas to outside of the system in a process for contacting to the ion-exchange resin or the solid catalyst. For example, when the sulfur compound(s) is a sulfite ester, the sulfite ester is converted to sulfurous acid gas by the desulfurization treatment method (1) or (2). However, the boiling point of the sulfurous acid gas is sufficiently low so as to be removed to a gas phase while the crude alcohol is contacted with the ion-exchange resin or the solid catalyst. In addition a detoxication treatment of the gas phase is necessary so that the sulfurous acid gas is not be released to the atmosphere.

When the desulfurization treatment is carried out by contacting the crude alcohol to the ion-exchange resin or the solid catalyst, it is preferable that the crude alcohol containing the sulfur compound(s) be diluted with water, and the obtained mixed solution be subjected to the desulfurization treatment. When the mixed solution, which is obtained by diluting a crude alcohol containing the sulfur compound(s) with water, is used, a part of the sulfur compounds react water. Thereby, the sulfur compound(s) may be converted to a compound which is easily desulfurized by the reaction process or a compound which is easily separated from the alcohol.

The desulfurization treatment with a physical adsorbent as the pre-desulfurization process is a method wherein sulfur compound(s) are physically absorbed to a suitable adsorbent to be removed. Examples of the adsorbent include activated carbon, activated white clay, diatom earth, silica, alumina, and zeolite.

The desulfurization treatment with a chemical adsorbent as the pre-desulfurization process is a method wherein sulfur compound(s) are physically absorbed to a suitable adsorbent to be removed. Examples of the chemical adsorbent include an ion-exchange resin, and cupper-based adsorbents.

In the desulfurization treatment with a physical adsorbent or desulfurization treatment with a chemical adsorbent, a method, in which the crude alcohol containing the sulfur compound(s) is continuously flown in a tower filled with the adsorbent, is used.

In this method, the temperature when the crude alcohol containing the sulfur compound(s) is in contact with the adsorbent is preferably in a range of 0° C. to 200° C., and more preferably in a range of room temperature (25° C.) to 100° C.

The contact temperature is preferably in a range of 0° C. to 200° C., because a detachment of the sulfur compound(s) adsorbed in the adsorbent does not easily occur, and adsorption efficiency is improved.

In the desulfurization treatment with a physical adsorbent or desulfurization treatment with a chemical adsorbent, after a certain content of the sulfur compounds is adsorbed in the adsorbent, the adsorbent cannot work. In this case, the adsorbent may be regenerated or new adsorbent is filled in the tower instead of the adsorbent which cannot work.

The separation process in the second embodiment of the method of producing an alcohol of this invention is a process in which a desulfurization process using a separation membrane based on a pervaporation method is applied to an alcohol desulfurized by a pre-desulfurization treatment.

The second embodiment of the method for producing an alcohol according to the present invention includes a pretreatment process in which the crude alcohol containing the sulfur compounds or the crude alcohol containing the sulfur compound(s) and 1 ppm by weight or more of methanol or propanols is subjected to at least one treatment selected from the desulfurization treatment by reaction process, the desulfurization treatment with a physical adsorbent, and the desulfurization treatment with a chemical adsorbent; and a separation process in which the alcohol which is desulfurized in the pretreatment process is desulfurized by using the separation membrane based on the pervaporation method.

According to the second embodiment of the method for producing an alcohol, it is possible to decrease the total sulfur content in the produced an alcohol preferably to less than 10 ppm by weight, more preferably to less than 1 ppm by weight, and most preferably to less than 0.5 ppm by weight. Therefore, it is possible to produce an alcohol which can be used as a raw material for a chemical process containing a catalytic reaction, fuel for automobiles, and other fuels.

Here, when the two processs, the pretreatment process and the separation process, are carried out, the production method is complicated as a desulfurization process. However, an alcohol can be produced with further improved efficiency than the first embodiment, depending on the kinds of the sulfur compound(s) to be desulfurized.

[Production Method for Hydrogen or Synthesis Gas]

The production method for hydrogen or a synthesis gas according to the present invention is a method in which the alcohol obtained by the method for producing an alcohol according to the present invention (the first and second embodiments) is subjected to a catalytic reforming process to produce hydrogen or synthesis gas.

The catalytic reforming process is one method for producing hydrogen or synthesis gas, and has many successful results to petroleum-based raw materials. In general, the catalytic reforming process includes a low temperature steam reforming reaction (pre-reforming) and a high temperature steam reforming reaction.

The high temperature steam reforming reaction is a reforming reaction wherein hydrocarbons and steam are mixed and they are reacted each other and reformed, normally, at 800° C. or more to obtain a synthesis gas.

The low temperature-steam reforming reaction is carried out to decrease load in the high temperature reforming reaction when the raw material contains many kinds of hydrocarbon. In the low temperature steam reforming reaction, steam is added and mixed with hydrocarbon to produce methane, etc under temperatures in a range of 250° C. to 550° C.

Ethanol is converted in synthesis gas containing mainly at least one of methane, carbon dioxide, hydrogen, and carbon monoxide or hydrogen by the low temperature steam reforming reaction in the first stage of the catalytic reforming process. The obtained synthesis gas or hydrogen can be used as an alternative petroleum fuel.

When the low temperature-steam reforming reaction is carried out without any problem, the high temperature-steam reforming reaction, which is carried out after the low temperature-steam reforming reaction, can be carried out easily.

[Alcohol]

An alcohol according to the present invention is the following an alcohol:

an alcohol obtained by subjecting the separation process in which an alcohol, which has the total sulfur content being preferably less than 10 ppm by weight, more preferably less than 1 ppm by weight, and most preferably less than 0.5 ppm by weight, and which contains 1 ppm by weight or more of methanol or propanols, is desulfurized by contacting to the separation membrane based on the pervaporation method to decrease the content of the sulfur compound(s) contained; or

an alcohol obtained by subjecting an alcohol which has the total sulfur content being preferably less than 10 ppm by weight, more preferably less than 1 ppm by weight, and most preferably less than 0.5 ppm by weight, and which contains 1 ppm by weight or more of methanol or propanols, to the pre-treatment process which is at least one selected from the group consisting of the desulfurization treatment by reaction process, the desulfurization treatment with a physical adsorbent, and the desulfurization treatment with a chemical adsorbent, and the separation process in which desulfurized an alcohol obtained in the pre-treatment process is desulfurized by contacting to the separation membrane based on the pervaporation method to decrease the content of the sulfur compounds contained.

In other words, the alcohol according to the present invention is an alcohol produced by the method for producing an alcohol according to the present invention as explained above (the first and the second embodiments).

Therefore, the alcohol according to the present invention is an alcohol useful as a raw material for a chemical process containing a catalytic reaction, fuel for automobiles, or other fuels.

### EXAMPLES

Below, the present invention is explained in more detail with reference to Examples and Comparative Examples. However, the present invention is not limited to the following Examples.

First, measuring methods, which were carried out in Examples and Comparative Examples, are explained.

(1) Measurement of Methanol and Propanols Contained in a Crude Alcohols

The concentration of methanol and propanols contained in the crude alcohol was measured using gas chromatography. The measuring conditions are shown in Table 1. The results of measurement are shown by “ppm by weight”.

TABLE 1

| Material to be measured | Methanol | Propanols |
|---|---|---|
| Machine model | GC-2010 | 5890 series II |
| Manufacturer | Shimadzu | HEWLETT |
| | Corporation | PACKARD |
| Column used | Ethyl vinyl | Polyethylene |
| | benzene-divinyl | glycol |
| | benzene copolymer | capillary |
| | capillary column | column |
| Injection temperature | 200° C. | 200° C. |
| Analysis initial temperature | 40° C. | 40° C. |
| Rate of temperature rise | 5° C./min. | 5° C./min. |
| Analysis final temperature | 200° C. | 200° C. |
| Detector and Temperature | FID and 210° C. | FID and 210° C. |
| Carrier gas and flow rate | Helium and | Helium and |
| | 15 mL/min. | 15 mL/min. |
| Content of sample injected | 1 μL | 1 μL |

(2) Measurement of the Concentration of Sulfur Contained in the Crude Alcohol

The concentration of the sulfur compound(s) contained in the crude alcohol (total content of sulfur) was measured using coulometry (TOX-100, marketed by DIA Instruments Co., Ltd.). The results of the measurement are shown by “ppm by weight based on sulfur”.

(3) Recovery Ratio of Process Liquid

The recovery ratio of the target alcohol was calculated as a weight distillate of the process liquid obtained relative to the weight of liquid subjected to the desulfurization treatment (the crude alcohol containing the sulfur compound(s)).

### Example 1

A crude alcohol containing ethanol as a target alcohol and the sulfur compound(s) was desulfurized using the desulfurization equipment shown in FIG. 1.

As the tube type separation membrane 13, a tube type silicone membrane (trade name: SR1554, marketed by Tigers Polymer Corporation) having an inner diameter of 1 mm, a thickness of 1 mm, and an effective length of 6 m was used.

As the crude alcohol, as shown in Table 2, an ethanol solution diluted with water (ethanol:water=1 mol:2 mol; and water content: 44% by weight) was prepared using non-desulfurized ethanol “ET-1” containing methanol, propanols, and the sulfur compound(s).

The ethanol solution diluted with water was passed inside of the tube type separation membrane 13 at room temperature with a flow rate of 1.57 mL/min. ((average) linear velocity of 0.83 cm/second; residence time: 120 minutes), and desulfurized solution 21 was obtained.

The concentration of the sulfur compounds in the desulfurized solution (containing water used for dilution) obtained was measured by the method explained in measurement of the concentration of sulfur compound(s) (Measurement of the total sulfur contant) above. In addition, the recovery ratio of a desulfurized solution obtained was also measured. These results are shown in Table 3.

### Example 2

Ethanol containing the sulfur compounds was desulfurized in a manner identical to that of Example 1, except that non-desulfurized ethanol ET-2 in Table 2 was used as the crude alcohol and that the residence time of the ethanol diluted with water in tube type separation membrane was adjusted to 60 minutes.

The concentration of the sulfur compound(s), and the recovery ratio of the obtained desulfurized solution (containing water for dilution) were measured as explained above. The results are shown in Table 3.

### Example 3

Ethanol containing the sulfur compound(s) was desulfurized in a manner identical to that of Example 1, except that non-desulfurized ethanol ET-3 in Table 2 was used as the crude alcohol.

The concentration of the sulfur compound(s), and the recovery ratio of the obtained desulfurized solution (containing water for dilution) were measured as explained above. The results are shown in Table 3.

### Example 4

Ethanol containing the sulfur compound(s) was desulfurized in a manner identical to that of Example 2, except that non-desulfurized ethanol ET-4 in Table 2 was used as the crude alcohol.

The concentration of the sulfur compound(s), and the recovery ratio of the obtained desulfurized solution (containing water for dilution) were measured as explained above. The results are shown in Table 3.

### Example 5

Ethanol containing the sulfur compound(s) was desulfurized in a manner identical to that of Example 1, except that non-desulfurized ethanol ET-5 in Table 2 was used as the crude alcohol, and that the residence time for ethanol diluted with water in tube type separation membrane was adjusted to 50 minutes.

The concentration of the sulfur compound(s), and the recovery ratio of the obtained desulfurized solution (containing water for dilution) were measured as explained above. The results are shown in Table 3.

### Example 6

Ethanol containing the sulfur compound(s) was desulfurized in a manner identical to that of Example 1, except that non-desulfurized ethanol ET-6 in Table 2 was used as the crude alcohol, and that the residence time for ethanol diluted with water in tube type separation membrane was adjusted to 30 minutes.

The concentration of the sulfur compound(s), and the recovery ratio of the obtained desulfurized solution (containing water for dilution) were measured as explained above. The results are shown in Table 3.

### Example 7

Ethanol containing the sulfur compound(s) was desulfurized in a manner identical to that of Example 2, except that non-desulfurized ethanol ET-7 in Table 2 was used as the crude alcohol.

The concentration of the sulfur compound(s), and the recovery ratio of the obtained desulfurized solution (containing water for dilution) were measured as explained above. The results are shown in Table 3.

### Example 8

Ethanol containing the sulfur compound(s) was desulfurized in a manner identical to that of Example 6, except that non-desulfurized ethanol ET-8 in Table 2 was used as the crude alcohol.

The concentration of the sulfur compound(s), and the recovery ratio of the obtained desulfurized solution (containing water for dilution) were measured as explained above. The results are shown in Table 3.

### Example 9

Ethanol containing the sulfur compound(s) was desulfurized in a manner identical to that of Example 2, except that non-desulfurized ethanol ET-9 in Table 2 was used as the crude alcohol.

The concentration of the sulfur compound(s), and the recovery ratio of the obtained desulfurized solution (containing water for dilution) were measured as explained above. The results are shown in Table 3.

### Example 10

Ethanol containing the sulfur compound(s) was desulfurized in a manner identical to that of Example 6, except that non-desulfurized ethanol ET-10 in Table 2 was used as the crude alcohol.

The concentration of the sulfur compound(s), and the recovery ratio of the obtained desulfurized solution (containing water for dilution) were measured as explained above. The results are shown in Table 3.

TABLE 2

| | Alcohol | Content of impurities (ppm by weight) | | |
|---|---|---|---|---|
| Alcohols | component | Methanol | Propanols | Total sulfur |
| ET-1 | Ethanol | 350 | 4582 | 49 |
| ET-2 | Ethanol | 124 | 8700 | 100 |
| ET-3 | Ethanol | 105 | 6739 | 79 |
| ET-4 | Ethanol | 57 | 875 | 34 |
| ET-5 | Ethanol | 55 | 834 | 32 |
| ET-6 | Ethanol | 54 | 821 | 9 |
| ET-7 | Ethanol | 54 | 536 | 48 |
| ET-8 | Ethanol | 52 | 513 | 5 |
| ET-9 | Ethanol | <1 | <1 | 44 |
| ET-10 | Ethanol | <1 | <1 | 2.1 |

In Table 2, “Alcohols” means the type of the crude alcohol containing the sulfur compound(s), and “Alcohol component” means target alcohol to be obtained.

TABLE 3

| | | Desulfurization treatment | | Sulfur content | |
|---|---|---|---|---|---|
| | | conditions | | after | |
| | | Residence | | desulfurization | Recovery |
| | | time | | treatment | ratio |
| Example | Alcohols | (min.) | Temperature | (ppm by weight) | (%) |
| 1 | ET-1 | 120 | Room temp. | Less than 0.5 | 93 |
| 2 | ET-2 | 60 | Room temp. | 1 | 97 |
| 3 | ET-3 | 120 | Room temp. | 1 | 93 |
| 4 | ET-4 | 60 | Room temp. | 1 | 96 |
| 5 | ET-5 | 50 | Room temp. | 0.9 | 97 |
| 6 | ET-6 | 30 | Room temp. | 0.8 | 98 |
| 7 | ET-7 | 60 | Room temp. | Less than 0.5 | 97 |
| 8 | ET-8 | 30 | Room temp. | Less than 0.5 | 99 |
| 9 | ET-9 | 60 | Room temp. | Less than 0.5 | 97 |
| 10 | ET-10 | 30 | Room temp. | Less than 0.5 | 99 |

In Table 3, “Alcohols” means the type of the crude alcohol containing the sulfur compound(s).

It is clear from the results of Examples 1 to 10 that the total sulfur content of the crude alcohol could be reduced to 1 ppm by weight or less by using the tube type separation membrane without depending on the sulfur content in the crude alcohol, the existence or nonexistence and concentration of methanol or propanols. In addition, it is also confirmed that the total sulfur content could be reduced to less than 0.5 ppm by weight depending conditions.

### Example 11

Butanol containing sulfur compound(s) was desulfurized in a manner identical to that of Example 1, except that the butanol was used as the crude alcohol without dilution with water and that the residence time of the butanol in the tube type separation membrane was adjusted to 90 minutes.

The concentration of the sulfur compound(s), and the recovery ratio of the obtained desulfurized solution were measured as explained above. The results are shown in Table 4.

### Example 12

Butanol containing the sulfur compound(s) was desulfurized in a manner identical to that of Example 2, except that non-desulfurized ethanol butanol containing the sulfur compound(s) was used as the crude alcohol.

The concentration of the sulfur compound(s), and the recovery ratio of the obtained desulfurized solution were measured as explained above. The results are shown in Table 4.

### Example 13

Butanol containing the sulfur compound(s) was desulfurized in a manner identical to that of Example 6, except that non-desulfurized ethanol butanol containing the sulfur compound(s) was used as the crude alcohol.

The concentration of the sulfur compound(s), and the recovery ratio of the obtained desulfurized solutionwere measured as explained above. The results are shown in Table 4.

TABLE 4

| | | Sulfur content before | Desulfurization treatment conditions | | Desulfurization Results | |
|---|---|---|---|---|---|---|
| | | desulfurization treatment | Residence | | Sulfur content | Recovery |
| Ex. | Alcohols | (ppm by weight) | time (min.) | Temp. | (ppm by weight) | ratio (%) |
| 11 | Butanol | 26.3 | 90 | Room | Less than 0.5 | 86 |
| | | | | temp. | | |
| 12 | Butanol | 6.3 | 60 | Room | Less than 0.5 | 90 |
| | | | | temp. | | |
| 13 | Butanol | 1.7 | 30 | Room | Less than 0.5 | 95 |
| | | | | temp. | | |

In Table 4, “Alcohols” means target alcohol to be obtained.

It is confirmed from the results of Examples 11 to 13 that the total sulfur content of the crude alcohol could be reduced to less than 0.5 ppm by weight by using the tube type separation membrane even when the target alcohol is butanol.

### Example 14

The desulfurized solutions of Examples 14-1 to 14-4 were obtained in a manner identical to Example 1, except that non-desulfurized ethanol ET-4 in Table 2 was used as the crude alcohol and that the residence time of ethanol diluted with water in the tube type separation membrane was varied to 10 minutes, 30 minutes, 60 minutes, and 120 minutes.

The concentration of the sulfur compound(s) of the obtained desulfurized solution (containing water for dilution) was measured as explained above. The results are shown in Table 5.

TABLE 5

| | | Desulfurization treatment | | Sulfur content |
|---|---|---|---|---|
| | | conditions | | after |
| | | Residence | | desulfurization |
| | | time | | treatment |
| Example | Alcohols | (min.) | Temperature | (ppm by weight) |
| 14-1 | ET-4 | 10 | Room temp. | 11 |
| 14-2 | ET-4 | 30 | Room temp. | 3 |
| 14-3 | ET-4 | 60 | Room temp. | 1 |
| 14-4 | ET-4 | 120 | Room temp. | Less than 0.5 |

In table 5, “Alcohols” means the type of the crude alcohol containing the sulfur compound(s).

It is confirmed from Table 5 that the content of sulfur compounds contained in ethanol which is the target alcohol could be further reduced by making the residence time of a crude alcohol diluted with water in the tube type separation membrane longer.

### Example 15-1

The desulfurized solution was obtained in a manner identical to Example 2, except that non-desulfurized ethanol ET-7 in Table 2 was used as the crude alcohol without dilution with water.

The concentration of the sulfur compound(s), and the recovery ratio of the obtained desulfurized solution (containing water for dilution) were measured as explained above. The results are shown in Table 6.

### Example 15-2

The desulfurized solution was obtained in a manner identical to Example 2, except that non-desulfurized ethanol ET-7 in Table 2 was used as the crude alcohol without dilution with water, and that the residence time for ethanol diluted with water in tube type separation membrane was adjusted to 90 minutes.

The concentration of the sulfur compound(s), and the recovery ratio of the obtained desulfurized solution (containing water for dilution) were measured as explained above. The results are shown in Table 6.

### Example 16-1

The desulfurized solution was obtained in a manner identical to that of Example 2, except that non-desulfurized ethanol ET-9 in Table 2 was used without dilution with water as the crude alcohol.

The concentration of the sulfur compound(s), and the recovery ratio of the obtained desulfurized solution (containing water for dilution) were measured as explained above. The results are shown in Table 6.

### Example 16-2

The desulfurized solution was obtained in a manner identical to that of Example 1, except that non-desulfurized ethanol ET-9 in Table 2 was used without dilution with water as the crude alcohol, and that the residence time for ethanol diluted with water in the tube type separation membrane was adjusted to 90 minutes.

The concentration of the sulfur compound(s), and the recovery ratio of the obtained desulfurized solution (containing water for dilution) were measured as explained above. The results are shown in Table 6.

TABLE 6

| | | Desulfurization treatment | | Sulfur content | |
|---|---|---|---|---|---|
| | | conditions | | after | |
| | | Residence | | desulfurization | |
| | | time | | treatment | Recovery |
| Example | Alcohols | (min.) | Temperature | (ppm by weight) | ratio (%) |
| 15-1 | ET-7 | 60 | Room temp. | 1.3 | 91 |
| 15-2 | ET-7 | 90 | Room temp. | Less than 0.5 | 88 |
| 7 | ET-7 | 60 | Room temp. | Less than 0.5 | 97 |
| 16-1 | ET-9 | 60 | Room temp. | 1.6 | 91 |
| 16-2 | ET-9 | 90 | Room temp. | Less than 0.5 | 88 |
| 9 | ET-9 | 60 | Room temp. | Less than 0.5 | 97 |

In Table 6, “Alcohols” means the type of a crude alcohol containing the sulfur compounds.

It is confirmed from Table 6 that the recovery ratio of the desulfurized solution is improved in addition that the efficiency of desulfurization is improved when a non-desulfurized crude alcohol containing ethanol as the target alcohol is desulfurized after dilution with water.

In other words, it is confirmed that when the residence time is the same (60 minutes), Example 7, in which the crude ethanol is diluted with water, has a higher recovery ratio than that of Example 15-1. In addition, when Examples 15-2 and 7 are compared, it is confirmed that the time for reducing the concentration of the sulfur compounds contained in ethanol to the same level (to less than 0.5 ppm by weight) is shorter in Example 7 than Example 15-2.

Furthermore, it is confirmed that when the residence time is the same (60 minutes), Example 9, in which the crude ethanol is diluted with water, has a higher recovery ratio than that of Example 16-1. In addition, when Examples 16-2 and 9 are compared, it is confirmed that the time for reducing the concentration of the sulfur compound(s) contained in ethanol to the same level (to less than 0.5 ppm by weight) is shorter in Example 9 than Example 16-2.

### Example 17

The crude alcohol containing ethanol as the target alcohol and the sulfur compound(s) was desulfurized using the desulfurization equipment shown in FIG. 2.

A hollow fiber type separation membrane 33 (total surface area: 0.55 m²; trade name NAGASEP M40-B, marketed by Nagayanagi Co., Ltd.), in which 6000 hollow fiber membranes which were made of silicone, and had the inner diameter: 0.17 mm, the outer diameter: 0.25 mm, and the effective length: 140 mm, were bound, was used.

Non-desulfurized ethanol ET-4 in Table 2 as an alcohol was circulated at 50° C. in a circuit of the vessel 31→the flow channel 37→the separator 34→the flow channel 39→the vessel 31→the flow channel 37→ . . . . At the same time, a part of the evaporated component which was evaporated by passing through the hollow fiber type separation membrane 33, was collected in the trap vessel 35 using nitrogen gas (1 L/min.). Then, the evaporated component collected was cooled to −195° C. and condensed using liquid nitrogen 60 to recover the condensation liquid of the evaporated component.

When the concentration of the sulfur compound(s) in the obtained condensation liquid was measured by the method explained above, the total sulfur content was 448 ppm.

From this result, it is confirmed that the content of the sulfur compounds contained in ethanol, which is the target alcohol, could be reduced by the desulfurization treatment in which the crude alcohol is contacted with a separation membrane based on a pervaporation method.

### Example 18

In the steam-reforming reaction of ethanol, the influences by the sulfur compound(s) contained in ethanol were examined.

The low temperature-steam reforming reaction of ethanol was carried using a reactor filled with a reforming catalyst, in a sand type fluid bed out under the conditions in which the temperature was 330° C., the reactor pressure was 1.5 MPaG, and a ratio (water/ethanol) between water and ethanol was 2.0 mol/mol.

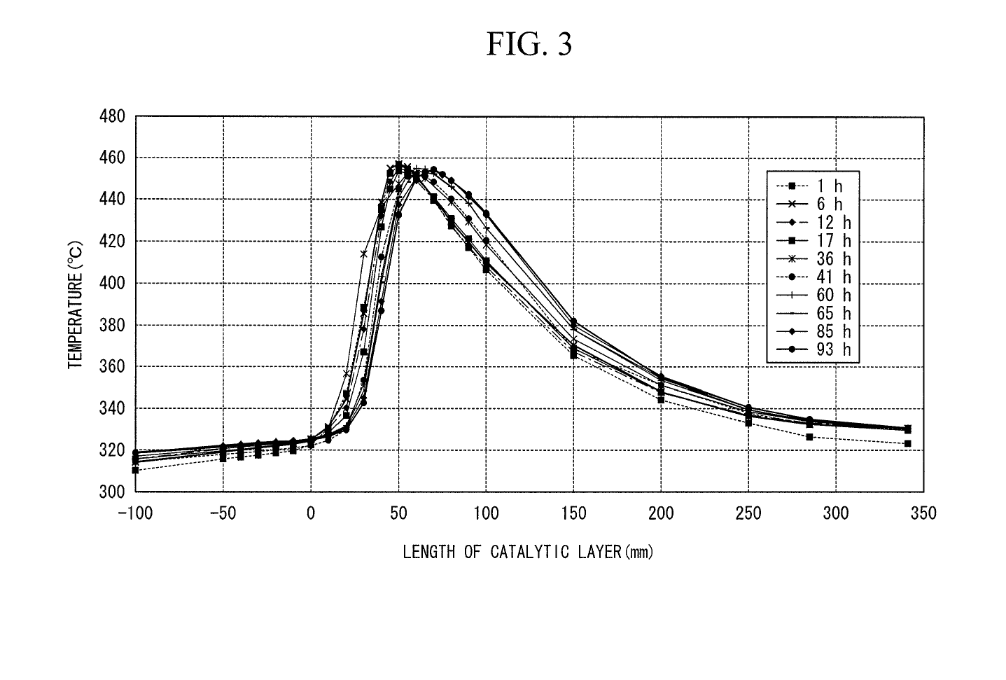

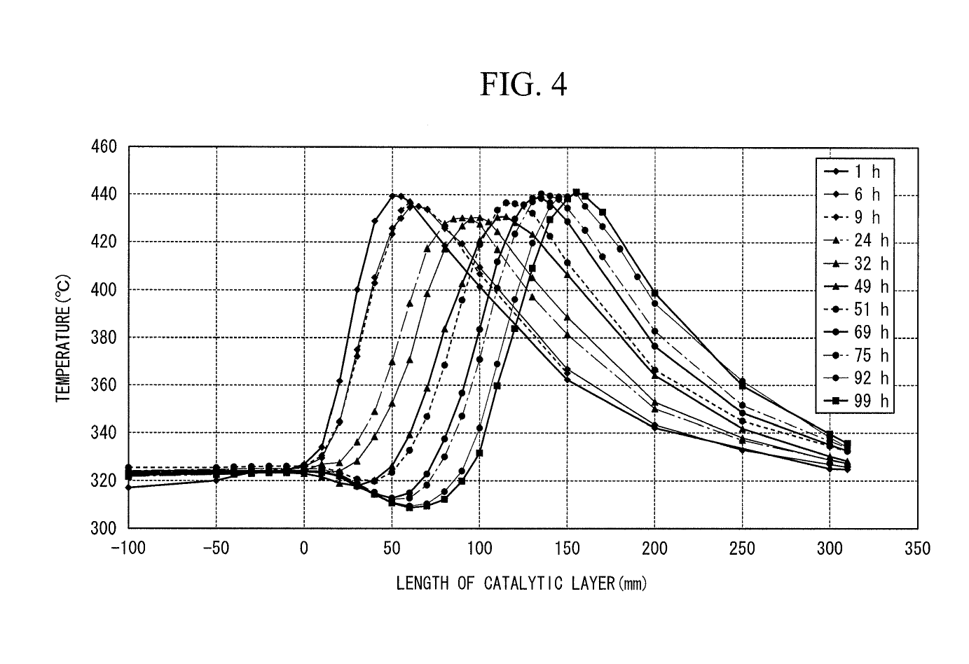

During the low temperature steam reforming reaction of ethanol, the time course of temperature distribution in the reactor was measured. The time course of temperature distribution in the reactor when ethanol containing almost no sulfur compounds was used is shown in FIG. 3. On the other hand, the time course of temperature distribution in the reactor when non-desulfurized ethanol ET-1 in Table 2 was used is shown in FIG. 4.

In the low temperature steam-reforming reaction of ethanol, the temperature of the reforming catalyst increases by the reaction heat. However, it is understood from FIGS. 3 and 4 that the heated place is hardly changed in the case of using the ethanol containing almost no sulfur compounds but the heating spot moves toward downstream in the case of using the ethanol containing the sulfur compound(s). This phenomenon may be caused because the sulfur compound(s) poison the reforming catalyst.

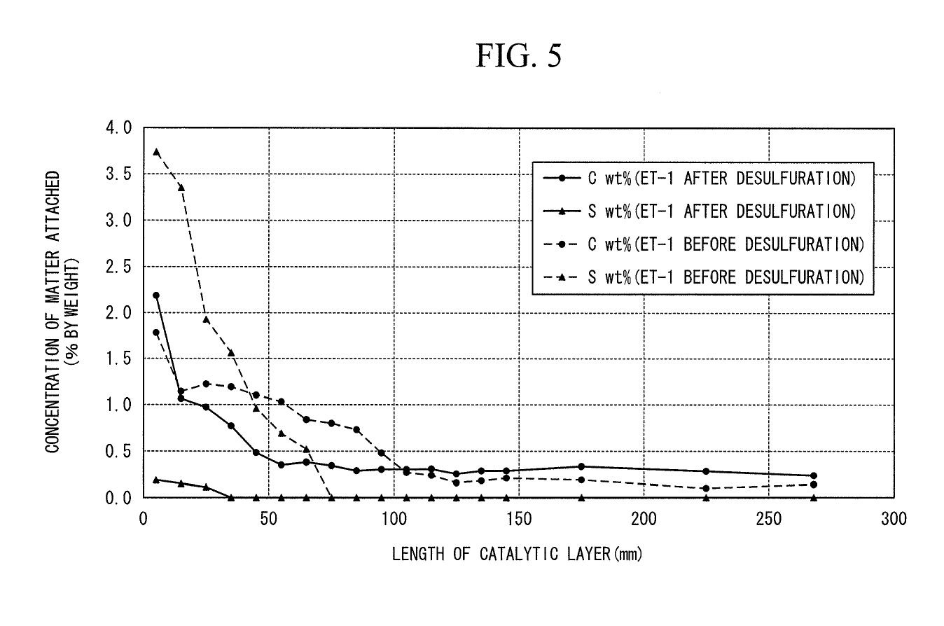

Therefore, after the test, the content of sulfur and carbon attached to the reforming catalyst used was measured, and the distribution of sulfur and carbon attached to the reforming catalyst as shown in FIG. 5 was confirmed.

As a result, it is confirmed that the phenomenon was caused by poisoning of the reforming catalyst by the sulfur compound(s). In addition, it is also confirmed that when the sulfur compound(s) is supplied to the reforming catalyst, the reforming catalyst adsorbs the sulfur compound(s) from the upstream, the reforming reaction became not to occur on the reforming catalyst, and soot is generated by thermal decomposition of an alcohol.

### Comparative Example 1

As a solution of non-desulfurized ethanol diluted with water, the solution which was prepared so that ethanol:water is 1 mol:2 mol (water content is 44% by weight) was used.

The ethanol diluted with water was desulfurized using a reactor in which a desulfurization treatment catalyst and an adsorbent are connected in a sand type fluid bed at 350° C., under the conditions in which the reactor pressure was 2.0 MPaG, in the presence of hydrogen, and a ratio (hydrogen/ethanol) between hydrogen and ethanol was 0.3 mol/mol.

The desulfurization treatment catalyst was CoO—MoO₃/γ-Al₂O₃ (below, abbreviated as “catalyst A”, trade name: CDS-LX1; marketed by JGC Catalyst and Chemicals Ltd.). The adsorbent was ZnO whose particle diameter is adjusted from 1.7 mm to 2.8 mm (below, abbreviated as “absorbent B”) using ZnO in which purity and content of alumina are different (ZnO purity: 89.0% by weight, alumina: 4.0% by weight).

Similar to Example 1, the total sulfur content of the treated solution (containing the dilution water) was measured. The total sulfur content was 45.7 ppm by weight or more. This shows that the desulfurization treatment was not carried out sufficiently.

### Comparative Example 2

Non-desulfurized ethanol diluted with water was desulfurized in a manner identical to that of Comparative Example 1, except that CoO—MoO₃/SiO₂ (below, abbreviated as “catalyst B”), which is a desulfurization treatment catalyst and has a support of SiO₂ having almost no acidity, and ZnO (adsorbent B) were used.

The total sulfur content of the treated solution (containing the dilution water) was measured. The total sulfur content was 43.3 ppm by weight or more. This shows that the desulfurization treatment was not carried out sufficiently.

### Comparative Example 3

Non-desulfurized ethanol diluted with water was desulfurized in a manner identical to that of Comparative Example 1, except that a desulfurization treatment catalyst (catalyst C), in which activated metal of cobalt and molybdenum are supported on supports containing y-alumina was used to react under conditions in which the temperature was 350° C., the reaction pressure was 2.0 MPaG, in the presence of hydrogen, and a ratio (hydrogen/ethanol) between hydrogen and ethanol was 0.2 mol/mol, and then a ZnO-based adsorbent catalyst (adsorbent C), which had been obtained by compressive molding (trade name: zinc oxide KCl grade, ZnO purity: 99% by weight or more, and alumina content: 0.0% by weight; marketed by Katayama Chemical Ltd.) such that the particle diameter be in a range of 1.7 mm to 2.8 mm, was used.

As a result of measuring the total sulfur content in the treated solution (containing water for dilution), the result was 44.5 ppm by weight or more. This shows that the desulfurization treatment was not carried out sufficiently.

It can be assumed from the results of Example 1 to 17 and Comparative Examples 1 to 3 that the alcohol contains a smaller content of the sulfur compound(s) than the alcohol obtained by desulfurization treatment using a conventional catalyst by the desulfurization treatment in which the crude alcohol is contacted with a separation membrane based on a pervaporation method.

### INDUSTRIAL APPLICABILITY

According to the method for producing an alcohol of the present invention, a method for producing the target alcohol which contains a remarkably low content of sulfur compound(s) by a simple desulfurization treatment from the crude alcohol which contains sulfur compound(s) can be provided.

### Explanation of Symbols

| | |
|---|---|
| 10: desulfurization equipment | 11: vessel |
| 12: pump | 13: tube type separation membrane |
| 14: recovery vessel | 15: flow channel |
| 20: a crude alcohol | 21: desulfurized solution |
| 30: desulfurization equipment | 31: vessel |
| 32: pump | 33: hollow fiber type separation membrane |
| 34: separator | 35: trap vessel |

36: low-temperature storing thermal insulation vessel

| | |
|---|---|
| 37, 38, and 39: flow channel | 40: inlet |
| 41: outlet | 42: gas inlet |
| 43: outlet | 50: a crude alcohol |
| 60: liquid nitrogen | |

1. A method for producing an alcohol comprising the steps of:
selecting a crude alcohol comprising a sulfur compound(s); and
submitting said crude alcohol to a separation process which reduces the content of said sulfur compound(s) through desulfurization treatment in which the crude alcohol is contacted with a separation membrane based on a pervaporation method.

2. The method for producing an alcohol according to claim 1, wherein the separation membrane is selected from the group consisting of a silicone membrane, a polyimide membrane, a polyamide membrane, a polyester membrane, and a polyvinyl alcohol membrane.

3. The method for producing an alcohol according to claim 1, wherein the separation membrane is a silicone membrane.

4. The method for producing an alcohol according to claim 1, wherein the crude alcohol contains at least one of methanol, 1-propanol, and 2-propanol, and the total content thereof is 1 ppm by weight or more.

5. The method for producing an alcohol according to claim 1, wherein the crude alcohol contains 20 ppm by weight or more of methanol.

6. The method for producing an alcohol according to claim 1, wherein the method reduces the total content of sulfur in the crude alcohol to less than 10 ppm by weight.

7. The method for producing an alcohol according to claim 6, wherein the method reduces the total content of sulfur in the crude alcohol to less than 1 ppm by weight.

8. The method for producing an alcohol according to claim 6, wherein the method reduces the total content of sulfur in the crude alcohol to less than 0.5 ppm by weight.

9. The method for producing an alcohol according to claim 1, wherein the crude alcohol contains 10 ppm by weight or more of the sulfur compound(s).

10. The method for producing an alcohol according to claim 1, wherein the crude alcohol is diluted with water and applied to the desulfurization treatment.

11. The method for producing an alcohol according to claim 1, wherein the crude alcohol is ethanol.

12. The method for producing an alcohol according to claim 1, wherein the method includes a pretreatment process conducted prior to the separation process in which the crude alcohol is subjected to at least one desulfurization treatment, selected from the group consisting of a reaction treatment physical adsorption, and chemical absorption.

13. A method for producing hydrogen or a synthesis gas, wherein the hydrogen or the synthesis gas is produced by subjecting the alcohol obtained by producing an alcohol according to claim 1, and subjecting said alcohol to a catalytic reforming reaction.

14. An alcohol obtained by the method for producing an alcohol according to claim 1.

15. The method for producing an alcohol according to claim 1, wherein the crude alcohol contains 200 ppm by weight or more of 1-propanol and/or 2-propanol in total.

16. The method for producing an alcohol according to claim 6, wherein the crude alcohol contains 10 ppm by weight or more of the sulfur compound(s).

17. The method for producing an alcohol according to claim 7, wherein the crude alcohol contains 10 ppm by weight or more of the sulfur compound(s).

18. The method for producing an alcohol according to claim 8, wherein the crude alcohol contains 10 ppm by weight or more of the sulfur compound(s).
